(19) United States
(12) Patent Application Publication
(10) Pub. No.: US 2025/0265767 A1
(43) Pub. Date: Aug. 21, 2025

(54) CO-REGISTRATION, DISPLAY, AND VISUALIZATION OF VOLUMETRIC SPECIMEN IMAGING DATA WITH PRE-SURGICAL IMAGING DATA

(71) Applicant: CLARIX IMAGING CORPORATION, Chicago, IL (US)

(72) Inventor: Xiao HAN, Chicago, IL (US)

(21) Appl. No.: 18/857,433

(22) PCT Filed: Apr. 19, 2023
(86) PCT No.: PCT/US2023/019070

§ 371 Date: Oct. 16, 2024

Related U.S. Application Data
(60) Provisional application No. 63/332,865, filed on Apr. 20, 2022.

(51) Int. Cl. G06T 15/20; G06T 3/60; G06T 5/50; G06T 7/10; G06T 7/30; G06V 10/74; G06V 20/70

(52) U.S. Cl. CPC G06T 15/20; G06T 3/60; G06T 5/50; G06T 7/10; G06T 7/30; G06V 10/761; G06V 20/70; G06T 2207/20221

(57) ABSTRACT

Embodiments are provided for improved imaging, analysis, and visualization of surgically explanted samples or other varieties of tissue sample in relation to image data depicting the samples prior to removal from the body. These embodiments facilitate analysis of the sample relative to the tissues pre-explantation, guiding the removal of additional tissue, determination of a diagnosis or prognosis, or performance of some other task or analysis. 3D images of an explanted sample are registered to target 2D or 3D images of the portion of the body that included the sample prior to sample explantation. The availability of 3D image data of the sample facilitates improved registration to the pre-explantation imagery', including by numerically projecting the 3D image to a target 2D image in order to determine the translation and orientation of the 3D sample needed to align to the target 2D image.

### CROSS-REFERENCE TO RELATED APPLICATION

This application calls priority to U.S. provisional application No. 63/332,865, filed Apr. 20, 2022, the contents of which are hereby incorporated by reference. The contents of U.S. Pat. No. 8,605,975, filed Oct. 11, 2010, U.S. application no. 2014/0161332, filed Dec. 2, 2013, U.S. Pat. No. 9,189,871, filed Dec. 29, 2014, U.S. Pat. No. 9,613,442, filed Nov. 5, 2015, International application no. US18/52175, filed Sep. 21, 2018, U.S. Provisional Patent Application No. 62/562,138, filed on Sep. 22, 2017, International Application PCT/US20/62462, filed Nov. 26, 2020, and International Application PCT/US21/20020, filed Feb. 26, 2021, are also hereby incorporated by reference.

### BACKGROUND

The treatment for a variety of health conditions can include the removal of specified tissues from the body. For example, treatment of certain cancers can include surgically removing one or more tumor masses from the body. Other conditions can be treated by removal of other types of tissue, foreign bodies, or other masses from the body. In performing such a removal, it is desirable to ensure complete removal of the target tissue while removing as little as possible of nearby healthy tissue. In practice, surgeons will often remove additional tissue around the target in order to ensure that the target is fully removed (e.g., to prevent relapse due to remnant tumor tissue continuing to grow).

To improve patient health outcomes, a pathologist can analyze the explanted tissue in order to determine whether the entire target has been removed, to determine a type of tissue that was explanted (e.g., to verify that an explanted target structure was a malignant tumor), to perform DNA sequencing or other analysis on the explanted tissue (e.g., to tailor systemic anti-cancer treatments), or to provide some other benefit to the patient and/or to the general treatment of illness. This can include sectioning the sample in order to visually, microscopically, or otherwise optically inspect a target within the tissue sample. This inspection may permit the pathologist to identify the type of tissue in the target (e.g., malignant cancerous tissue, benign tumor tissue), a status of the target (e.g., likely to be pre or post metastatic), and whether the target was fully removed as a result of explantation of the tissue sample (e.g., by observing how close to a margin of the tissue sample the target tissue extends). The pathologist can then provide a final diagnosis as to the success of the target removal procedure, which may then be used to decide whether to perform an additional procedure to remove portions of the target that may remain in the patient.

### SUMMARY

An aspect of the present disclosure relates to a method including: (i) obtaining a target two-dimensional (2D) image of a portion of a body; (ii) obtaining a three-dimensional (3D) image of a sample explanted from the portion of the body; (iii) determining a registered translation and orientation of the 3D image such that the 3D image is aligned with the perspective of the portion of the body represented in the target 2D image; (iv) based on the registered translation and orientation, projecting the 3D image via numerical methods to the plane of the target 2D image, thereby generating a projected 2D image; and (v) displaying an indication of the projected 2D image overlaid on the target 2D image.

Another aspect of the present disclosure relates to a method including: (i) obtaining a target three-dimensional (3D) image of a portion of a body; (ii) obtaining a sample 3D image of a sample explanted from the portion of the body; (iii) determining a registered translation and orientation of the sample 3D image such that explanted tissue represented in the sample 3D image is aligned with tissue of the portion of the body represented in the target 3D image; and (iv) displaying an indication of the sample 3D image rotated and translated according to the registered translation and orientation overlaid on the target 3D image.

Yet another aspect of the present disclosure relates to a transitory or non-transitory computer-readable medium configured to store at least computer-readable instructions that, when executed by one or more processors of a computing device, causes the computing device to perform controller operations to perform the method of any of the above aspects.

Yet another aspect of the present disclosure relates to a system including: (i) a controller comprising one or more processors; and (ii) a transitory or non-transitory computer-readable medium having stored therein computer-readable instructions that, when executed by the one or more processors of the controller, cause the system to perform the method of any of the above aspects.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation.

### BRIEF DESCRIPTION OF THE FIGURES

FIG. 1A depicts a 2D image of a portion of a body, according to example embodiments.

FIG. 1B depicts a 3D image of a sample explanted from the portion of the body depicted in FIG. 1A, according to example embodiments.

FIG. 1C depicts the 3D image of FIG. 1B after having been rotated, according to example embodiments.

FIG. 1D depicts the 3D image of FIG. 1C after having been projected to a 2D image, according to example embodiments.

FIG. 2 depicts the 2D images of FIGS. 1A and 1D overlaid on each other, according to example embodiments.

FIG. 3A depicts a 2D image of a portion of a body, according to example embodiments.

FIG. 3B depicts the 2D image of FIG. 3A, with the extent of a 2D projection of a 3D image of a sample explanted from the portion of the body depicted in FIG. 3A overlaid thereon, according to example embodiments.

FIG. 4 is a simplified block diagram showing some of the components of an example system.

FIG. 5 is a flowchart of a method, according to an example embodiment.

FIG. 6 is a flowchart of a method, according to an example embodiment.

### DETAILED DESCRIPTION

Examples of methods and systems are described herein. It should be understood that the words “exemplary,” “example,” and “illustrative,” are used herein to mean “serving as an example, instance, or illustration.” Any embodiment or feature described herein as “exemplary,” “example,” or “illustrative,” is not necessarily to be construed as preferred or advantageous over other embodiments or features. Further, the exemplary embodiments described herein are not meant to be limiting. It will be readily understood that certain aspects of the disclosed systems and methods can be arranged and combined in a wide variety of different configurations.

### I. OVERVIEW

A variety of clinical interventions involve the removal of tumors or other undesirable tissue or substances. Ideally, only the unwanted tissue would be removed, sparing neighboring tissue. This is difficult to achieve in practice, so surgeons will often excise more tissue than is necessary so as to prevent leaving any of the unwanted tissue in the body where it can, e.g., lead to relapse. However, this must be balanced in order to spare healthy tissue, so as to improve post-operative outcomes with respect to tissue function and appearance. To facilitate this targeting, surgical procedures can be guided by pre-surgical imaging of the site of the tumor or other target tissue. For example, a mammogram may be taken of a breast and used to plan the removal of a tumor or other target tissue from the breast while minimizing removal of other tissues, avoiding vasculature, nerves, ducts, or other tissue, or taking into account other factors. The target tissue and/or surrounding tissue may also have inserted therein sutures, clips, staples, biopsy markers, fiducials, or other artifacts that may be used to facilitate a surgeon orienting the pre-surgical imagery to the anatomy during the surgical procedure. Such artifacts may be implanted for the express purpose of allowing the surgeon to orient the pre-surgical imagery to the anatomy and/or for some other purpose.

Tissue explanted during a surgery is often analyzed by a pathologist, surgeon, or other healthcare professional in order to determine whether the procedure was successful in fully removing a tumor or other target tissue/structure. The pathologist's diagnosis can then be used to determine whether to perform an additional procedure (to remove additional tissue), where to remove such tissue (e.g., at a location in the patient's body corresponding to a location of the tissue sample at which a tumor approaches and/or clearly exceeds the boundary of the tissue sample), or other healthcare decisions. It would be beneficial to perform such imaging and analysis quickly after explantation of the sample, so as to guide the removal of additional tissue during a single surgical procedure. This avoids the cost and risks of performing an additional separate procedure (e.g., avoiding the risks associated with anesthesia) as well as avoiding the possibility of metastasis or other unwanted disease progression due to the additional time that remnant tumor cells would remain in the body. Additionally, it is desirable to generate imaging data for the explanted tissue sample so as to compare the same image data with the pre-surgical image data. This could allow the surgeon, radiologist, or other healthcare professional to compare the tissue removed to the pre-surgical plan in order to establish whether all of a target region of the anatomy has been removed, and if not, from which margin of the sample site to remove additional tissue in order to completely accomplish the pre-surgical image-based surgical plan.

Embodiments described herein provide a variety of benefits, including benefits that address the above issues. These embodiments include scanning of explanted tissue samples to generate 3D image data (e.g., volumetric density information generated by a micro-CT imager) that can then be registered to pre-surgical images (e.g., 2D mammograms, tomosynthetic image data, 3D image data). Such scanning can be accomplished quickly, by an imager located in the operating suite, allowing the sample image data to guide additional tissue removal during the same procedure, thereby avoiding re-operation. Additionally, such 3D sample image data can be manipulated quickly in a variety of ways to accurately register the 3D image with pre-operative 2D (or 3D) images.

The registered 3D image data can then be processed and displayed with the pre-operative imagery to allow the surgeon, radiologist, or other healthcare professional to make better decisions about the completeness of the surgical procedure, the need and location of additional tissue removal, and/or whether and how any tissue removed comports with a pre-surgical plan. For example, the registered 3D image can be numerically projected to the same plane as a pre-surgical 2D image of the anatomy from which the sample was taken and the images displayed overlaid on each other, allowing a surgeon to compare their conception of the extent of a tumor or other target tissue with the extent and contents of the sample that has actually been removed from a patient's body. This can allow the surgeon to, e.g., determine that an area annotated on the pre-operative 2D image as containing a target tumor or other target tissue extends past the margin of the sample as removed from the patient's body, thus necessitating the removal of additional tissue from the patient's body, along the region of the surgical margin corresponding to the location in the overlaid images where the annotation area extends beyond the explanted sample image.

The use of 3D explanted sample image data facilitates improvements in the identification and localization of sutures, clips, fiducials, or other artifacts within the sample in order to help a surgeon or other healthcare professional in determining the relationship of the sample with pre-surgical procedures (e.g., biopsy, palpation, etc.). The high-resolution 3D image data that is available when a sample is imaged separately outside of the body allows the location and orientation of such fiducials to be determined more accurately. Such improved localization accuracy can lead to improved registration of the 3D image data to a 2D (or 3D) pre-surgical image, e.g., by determining the location and orientation of the fiducial within the pre-surgical image and then registering the pre-surgical image and sample image based on the determined location and orientation of the fiducial within each of the images.

It should be understood that the above embodiments, and other embodiments described herein, are provided for explanatory purposes, and are not intended to be limiting.

### II. EXAMPLE EMBODIMENTS FOR IMAGE-GUIDED SAMPLE EXPLANTATION

As briefly noted above, it is desirable to be able to directly compare pre-surgical images of a surgical site (e.g., of a breast that contains a tumor or other tissue to be removed) with 3D images of any tissue sample(s) removed therefrom. This can facilitate improved assessment of the completeness of the procedure in removing any target tissues identified within the pre-surgical image(s) (and if incomplete, improved determination of what additional remnant tissue to remove in a revision procedure in order to complete removal of the target tissue). This can also, in embodiments wherein the sample image data is generated during the sample explantation surgery, guide the surgeon in removing such additional tissue, improving patient outcomes by ensuring more complete target tissue removal and reducing the chance that additional tissue removal procedure(s) will be needed.

Accordingly, the embodiments described herein provide the means to generate high-quality 3D images of explanted tissue samples (e.g., based on volumetric density information of the samples as generated by a micro-CT imager) and to accurately register such 3D images to 2D (or 3D) pre-surgical images of target tissue within a region of the body from with the tissue samples were removed. The availability of 3D images of the explanted samples means that a variety of analyses and visualizations of the sample can be provided (e.g., numerically projected 2D views of the sample from a variety of angles, slices through the sample at a variety of angles/locations). Additionally, such 3D image data allows the 3D image to be registered to a 2D pre-surgical target image regardless of the angle/plane of the 2D image (e.g., as compared with taking one or a few 2D images through the sample, where it is unlikely that the angle/plane of any one of the available images closely matched the plane/angle of the 2D target image).

Note that a variety of embodiments are described herein wherein anatomical images (e.g., 2D pre-surgical images of a target surgical site, images or renders of a 3D image of a sample surgically removed from such a target site) are displayed to a surgeon, radiologist, or other healthcare professional. In some examples, such a display is provided by a computing system (e.g., an imaging system) that operates an imager to generate scan data for a sample, that reconstructs volumetric density information from the scan data, that registers 3D images generated therefrom to pre-surgical 2D (or 3D) images, and that renders two-dimensional images and/or generates other analyses based on the image data. However, such a display can also be part of a pathologist's workstation, a remote control and display unit for an imaging system, or some other interface system that does not operate to reconstruct volumetric density information for a sample or to render two-dimensional or other types of images or image information therefrom.

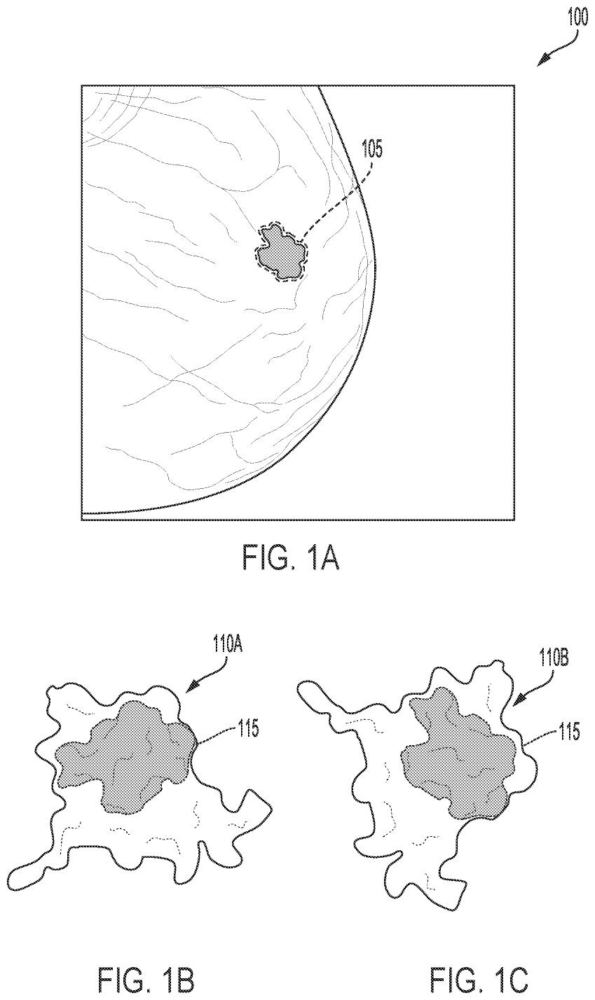

FIG. 1A depicts an example target 2D image 100 of a portion of a body (e.g., a breast). FIG. 1B depicts aspects of an example 3D image 110A (e.g., a 3D matrix of volumetric density values) of a sample explanted from the portion of the body depicted in the target 2D image 100. The embodiments described herein facilitate the registration of the 3D image 110A to the target 2D image 100 such that the contents of the images can be displayed together (e.g., overlaid). Such a combined display can enable a surgeon or other healthcare professional to evaluate the success of the explantation procedure, plan the removal of additional tissue (e.g., during the same procedure as during which the original sample was removed), determine a disease prognosis, decide on a plan of treatment (e.g., whether to provide chemotherapy, a dose of chemotherapy), or perform some other analysis.

Such a combined display of the pre-surgical target 2D image and the 3D sample image or other image data derived therefrom could take a variety of forms. For example, the 3D image data could be represented by a semi-transparent rendering (e.g., of the boundaries of the sample and optionally of one or more regions of interest therein, of the volumetric density or other 3D information represented by a simulated fog or other occlusion) that surrounds the target 2D image, allowing the combination to be rotated to see the combined image data from different directions and/or to adjust the location of the target 2D image relative to the 3D image normal to the plane of the 2D image. Additionally or alternatively, the 3D image could be projected (e.g., via numerical methods) to the plane of the target 2D image and the two 2D image display side-by-side, or overlaid on each other (e.g., with the different images depicted by different colors, allowing similarities and differences between the images to be readily apprehended).

A variety of different methods could be used to generate volumetric density information or other 3D image data for a tissue sample. For example, a micro-CT imager can be used to generate X-ray radiopacity density information, an MRI imager can be used to generate hydrogen atom or MRI contrast density information, etc. In order to use such 3D imagery to, e.g., compare to pre-surgical 2D imagery, to determine whether a revision surgery is indicated, etc. it is generally advantageous to render, from the 3D image, one or more two-dimensional images of the sample. Such in two-dimensional images can include high-resolution cross-sectional images of slices through the sample, e.g., slices through the sample that are parallel to the standard coronal, sagittal, and/or transverse planes of the sample according to the orientation of the sample within a patient's body and/or that are parallel to the plane of the pre-surgical target 2D image. Two-dimensional images can also include perspective views of the sample. Such perspective views could be useful to illustrate the orientation and location of high-resolution cross-sectional images relative to the sample. Additionally, such perspective views may show, in three-dimensional space, the location of tumors, staples, wires, or other substances or structures of interest within the tissue sample. In yet another example, such 2D images could be numerically generated 2D images that project the 3D image into a specified plane (e.g., the same plane as the target 2D image) as though the sample had been 2D-imaged in that specified plane.

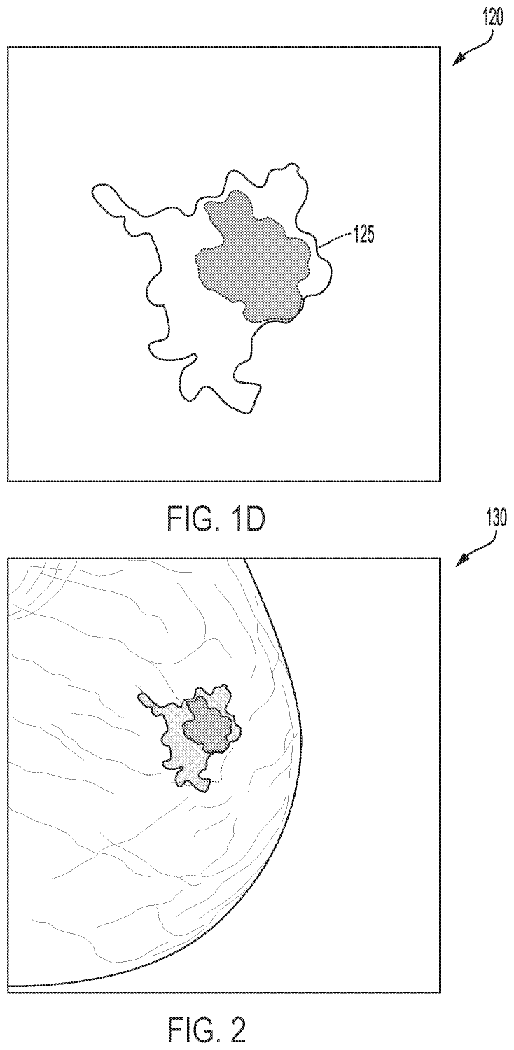

FIG. 2 depicts an example of the display of such a 2D numerical projection 120 of the registered 3D image 110B overlaid on the target 2D image 100. If annotations are available for either the target image and/or the 3D image, indications of such annotations can also be provided on the display. For example, a target annotation 105 of a target region within the target 2D image 110 is provided, as is a 3D region of interest 115 segmentation map within the 3D image 110A. The 3D region of interest 115 has also been projected as a two-dimensional region of interest 125 within the projected 2D image 120. Both of these annotations 105, 125 are also indicated on the overlaid 2D display depicted in FIG. 2.

Such annotations (in two or three dimensions) can be obtained in a variety of ways. In some examples, such annotations could be generated manually by a radiologist or other healthcare professional. For example, a radiologist could manually mark an area of a mammogram or other pre-surgical 2D image (e.g., 100) as being of interest (e.g., as containing a tumor to be removed from a breast). Additionally or alternatively, such annotations could be determined in an automated or semi-automated manner based on 2D or 3D images. For example, 3D images (e.g., rendered from volumetric imaging data of a tissue sample) could be used to identify regions of interest within the 3D image (e.g., regions of increased density that are likely to be tumors, calcifications, or other unwanted tissue). Indications of such identified regions of interest (e.g., segmentation maps indicating the location, extent, and/or geometry of such regions) may then be provided to a radiologist's workstation in order to, e.g., identify the location, extent, or other information about regions of interest within 2D or 3D images.

A segmentation map for tumors, staples, or other regions of interest within a sample could be generated in a variety of ways. In some examples, an automated algorithm could generate the segmentation map. This could include applying a density threshold to the volumetric density information (e.g., to segment staples, wire, calcifications, or other high-density content within the sample), applying a trained neural network, or performing some other process on the volumetric density information or other 3D or 2D image information. In some examples, the segmentation map could be generated by a radiologist or other healthcare professional (e.g., a pathologist). For example, the radiologist could annotate the extent of a tumor or other structure of interest within a tissue sample by, e.g., indicating the extent of the structure of interest in one or more two-dimensional cross-sectional images of the sample. A pathologist could be augmented by one or more automated segmentation methods. For example, an automated method could generate an estimated segmentation map which the pathologist could then edit (e.g., by dragging the edges of the segmentation map to expand or contract the volume of the sample that is included within the segmentation map). In another example, an automated method could generate a number of possible segmentations, and a pathologist could select the ‘best’ one.

Multiple different tumors, staples, wires, or other objects or sets of objects within a sample could be associated with respective different segmentation maps and/or corresponding regions of interest. A user interface could then provide a user with the ability to selectively blank certain contents of the sample from view by selecting or de-selecting the corresponding segmentation maps. A user selecting or deselecting individual objects or other contents within a sample for display in the manner could include clicking on or otherwise interacting with buttons of a user interface that are associated with respective contents of the sample, clicking on or otherwise interacting with portions of a display that are displaying contents of the sample, or interacting with a user interface in some other manner.

A 3D image of an explanted sample could be registered to a pre-surgical target 2D image of a portion of a body from which the sample was explanted in a variety of ways. The three-dimensional nature of the 3D image of the explanted sample allows a variety of transformations and projections to be applied thereto in order to facilitate registration and alignment to a target 2D image or to facilitate some other analysis or visualization. For example, the 3D image 110A could be rotated according to a candidate orientation to generate a rotated 3D image 110B, as depicted in FIG. 1C. This rotated 3D image 110B can then be projected, via numerical methods, to generate a candidate projected 2D image 120. The candidate projected 2D image 120 can then be aligned with the target 2D image 100 by employing a similarity metric between the target 2D image and versions of the candidate projected 2D image 120 translated in various ways (e.g., searching an enumerated set of vertical and horizontal translations, performing a gradient descent with respect to the translation based on the similarity metric) to determine a candidate translation of the candidate projected 2D image 120. The value of the similarity metric for the determined candidate translation could then be used as a similarity score for the candidate orientation, in order to compare different candidate orientations. Such a projection and translation can be determined for a plurality of different candidate orientations (e.g., for a regularly spaced grid of candidate orientations) and the corresponding similarity scores for each candidate orientation used to determine the correct orientation (and corresponding translation) to register the 3D image 110A to the target 2D image 100.

A variety of different numerical methods could be employed to project the 3D image to the plane of the target 2D image (or some other plane). This could include performing a plurality of numerical integrations (e.g., line integrals, volume integrals along cylinders, cones, or other geometry) through the 3D image (e.g., integrating the density information represented by the 3D image along lines, cones, or other paths through the 3D image) to determine the intensity or other value of pixels of the numerically projected 2D image. The specific paths/lines/locations/geometry of such integrals could be based on information about the equipment used to generate the target 2D image, in order to generate a projected 2D image that accurately simulates how the equipment used to generate the target 2D image would have imaged the explanted sample represented in the 3D image. For example, the geometry of the line integrals or other integral geometry for each pixel of the projected 2D image through the 3D image could be specified based on the relative location of an X-ray emitter and respective pixels of an X-ray sensor array of the equipment used to generate the target 2D image.

A variety of similarity metrics could be used to align the candidate projected 2D images 210 to the target 2D image 100, thereby determining similarity scores for their respective candidate orientations and allowing a final orientation to be determined to register the 3D image 110A to the target 2D image 100. For example, mutual information, Kullbeck-Leibler divergence, L2 norm, L1 norm, or some other metric(s) could be used. The mutual information between two images could be represented as

$$MI ( X ; Y ) = {\sum }_{y ∈ Y} {\sum }_{x ∈ X} {P}_{( X , Y )} ( x , y ) log ( \frac{{P}_{( X , Y )} ( x , y )}{{P}_{( X )} ( x ) {P}_{( Y )} ( y )} )$$

where MI( ) is the mutual information operator, X and Y are the images whose mutual information is to be computed, P_{(X,Y)}(x,y) is the joint probability mass function of the two images with respect to their pixel values (e.g., intensities), P_{(X)}(x) is the marginal probability mass function of the first image with respect to its pixel values, and P_{(Y)}(y) is the marginal probability mass function of the second image with respect to its pixel values.

The use of mutual information as the similarity metric for image comparison provides a variety of benefits. For example, the histogram borders and ‘bucket’ assignment of each pixel of the target 2D image 100 can be pre-computed once and re-used to determine the mutual information similarity metric against a variety of different candidate projected 2D images 120 and translations thereof. Further, the histogram borders and ‘bucket’ assignment of each pixel of individual candidate projected 2D images 120 can also be pre-computed once and re-used for the various attempted translations of the candidate projected 2D images 120 relative to the target 2D image 100. Yet further, determination of the mutual information in this way only requires counting the membership of any particular pair of pixels in the target 2D image 100 and a translation of a candidate projected 2D image 120, a computational savings relative to L2 distance or other similarity metrics which may require determination of nonlinear functions (e.g., power functions), sums of floating point numbers, or other computational tasks that are more complex, and thus more computationally costly, than counting the members of the various two-dimensional ‘buckets’ to generate the mutual information.

As noted above, an orientation can be determined to register a 3D image (e.g., 110A) with a target 2D image (e.g., 100) by assessing a plurality of candidate orientations (e.g., a pre-specified set of candidate orientations that spans all 4 pi steradians of solid angle) and then selecting the orientation that results in the greatest value of a similarity metric between the target 2D image and the 3D image as rotated according to the candidate orientation, projected into a 2D image, and then translated relative to the target 2D image in a manner that maximizes or otherwise results in an increased value of the similarity metric. Such a method of determining the registration orientation provides a variety of computational benefits, since the computation to assess of any one of the candidate orientations is independent of the computations for any other. Accordingly, the process of assessing all of the candidate orientations can be extensively parallelized in order to reduce the total time to evaluate the entire space of possible candidate orientations and to allow the amount of computational resources devoted to the task (e.g., number of GPUs or other processors, clock speed, processor power) to be throttled in order to obtain a desired total time to assess the set of candidate orientations. For example, less computational resources could be devoted to perform such a registration for post-surgical assessment, while more resources could be allocated in examples wherein the results of the registration are to be used intra-operatively to guide additional tissue removals and/or to terminate the surgical procedure.

The process of determining the registration orientation could be improved by searching a coarser ‘grid’ of candidate orientations, and then refining the orientation estimate determined at that cores resolution by performing a subsequent orientation search at a higher angular resolution in the neighborhood of the orientation determined by the coarse search. This could allow a higher-resolution registration orientation to be determined using less computational resources. Such a process could be performed at three or more progressively finer levels of search angular resolution.

Another way to reduce the computational cost to register a 3D image to a target 2D (or 3D) image is to limit the similarity assessment to only high-density (or otherwise high-intensity) contents of the images. For example, if the images contain clips, sutures, fiducials, or other artificial objects (e.g., objects placed in the tissue to facilitate the surgeon orienting pre-surgical imagery relative to the anatomy during a procedure), then the registration process could be based only on pixels of the target 2D image and/or 3D image that have values (e.g., intensity values) that exceed a threshold to select for such contents. Such a threshold could be specified such that pixels of the target 2D (or 3D) image and 3D image (or candidate projected 2D image determined therefrom) are discarded that do not depict metallic matter, ceramic matter, synthetic polymeric matter, or radiopaque matter. Additionally or alternatively, the threshold could be set to include pixels that represent calcifications or other high-intensity biological contents of the image(s). Such a threshold could be specified such that pixels of the target 2D (or 3D) image and 3D image (or candidate projected 2D image determined therefrom) are discarded that do not depict metallic matter, ceramic matter, synthetic polymeric matter, radiopaque matter, or calcifications.

The pixels of the image(s) that do not exceed the threshold could then be discarded and the registration process outlined above (or some other registration process) could proceed based only on the non-discarded pixels (treating the discarded pixels as ‘empty’ and/or zero-valued and thus avoiding computations related thereto as part of the registration process). The computational cost of the registration process could be further reduced by determining the location of a centroid or other measure of the location of the non-discarded pixels and beginning the registration process for a given orientation by starting with a candidate translation that corresponds the centroid of the projected 2D image to the centroid of the target 2D image.

The registration could be further improved, following the determination of a rotation and translation of the 3D image to correspond with the target 2D (or 3D) image, by deforming the rotated and translated 3D image (or projected 2D image determined therefrom). Such a deformation process could be performed to account for deformation of the sample following explantation and/or to account for deformation of the anatomy from which the sample was taken during generation of the target 2D (or 3D) image (e.g., to account for compression or other manipulation of a breast in order to generate 2D, tomographic, and/or 3D pre-surgical mammographic imagery).

Additionally or alternatively, the location and orientation of a fiducial in the images could be determined directly, and the registration of the 3D image to the target 2D image determined as the orientation and translation necessary to align the fiducial as depicted in the 3D image to the fiducial as depicted in the target 2D image. Such a method could be computationally cheaper, particular in examples wherein the fiducial is significantly denser or otherwise higher-intensity within the images, allowing the low-density (e.g., sub-threshold) pixels of the images to be discarded prior to determining the orientation and location of the fiducial within the images.

Such a fiducial could be an artificial radiopaque element or other feature to facilitate imaging via micro-CT, MRI, or some other volumetric means. Such a fiducial would thus be represented in the volumetric density information for a tissue sample, thereby allowing for registration of the volumetric density information with the physical location and extent of the sample receptacle. To facilitate the determination of the orientation of the fiducial, the fiducial could have a non-degenerate geometry, e.g., the fiducial could have a geometry that does not exhibit any axes of rotational symmetry and/or planes of symmetry. For example, the fiducial could have a tetrahedral the lengths of whose sides are all different. Alternatively, the fiducial could have a geometry that was relative non-degenerate (e.g., having no axes of rotational symmetry, but a single plane of symmetry) with any ambiguities in the determined orientation of the fiducial determined based on similarity between the overall image when evaluated according to the different ambiguous options for orientation, or relying on manual selection between the different orientation options.

Regardless of the manner of registration (e.g., assessment of candidate orientations and translations, determination of the location and orientation of fiducials), the registration process can include human input or feedback in order to improve the registration of the 3D image to the target 2D (or 3D) image. This could include determining multiple candidate registrations between a 3D image and a target 2D (or 3D) image and displaying indications of each of the candidate registrations to a user (e.g., displaying a projected 2D image of the 3D image overlaid on the target 2D image according to each of the candidate registrations). The user could then select which of the candidate registrations to use as the ‘true’ registration. The set of candidate registrations could be selected by, e.g., determining a set of candidate registrations that represent local maxima of the similarity metric with respect to candidate orientation. The set of such candidate registrations could be a set number (e.g., three candidate registrations are always provided) or selected according to some other consideration (e.g., only local maximum candidate registrations having similarity metric scores within a set percent of the absolute maximum similarity metric score). Additionally or alternatively, user input into the registration process could include the user providing modifications to the orientation and translation of a candidate registration (e.g., the user could ‘fine-tune’ an automatically generated registration determined using the methods described herein).

As noted above, display of the target 2D image, projected 2D image determined by projecting the 3D image into the plane of the target 2D image following registration of the 3D image, and/or displays of such imagery overlaid on each other could include indications of annotations that are associated with the imagery. Such annotations can also be further analyzed and the results of such analysis indicated on a display. For example, a region of overlap between annotated regions in the target 2D image and projected 2D image of the 3D image (e.g., an annotated region determined as a projection of a segmentation map within the 3D image) could be indicated on a display (e.g., on a display that is also indicating one or both of the target 2D image and projected 2D image). Additionally or alternatively, regions of the annotation in the target 2D image (which could indicate regions that a radiologist has identified as part of a tumor to be removed) that do not overlap with the extent of the explanted sample within the projected 2D image could be indicated on a display (e.g., to indicate portions of the target tumor that were not removed when the sample was explanted).

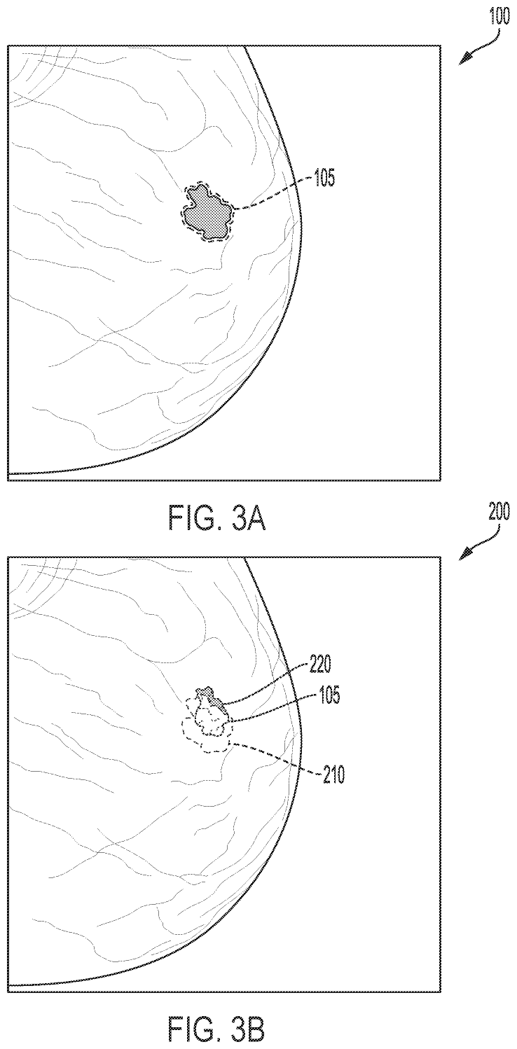

FIGS. 3A and 3B illustrate such a display. FIG. 3A depicts a target 2D image 100 with a target annotated region 105 indicated thereon. FIG. 3B depicts an overlaid display 200 of the target 2D image 100 and a projected 2D image that has been derived from a 3D image of a sample explanted from the region of the body depicted in the target 2D image. The extent of the explanted sample in the projected 2D image could be determined (e.g., by comparing the intensity of pixels of the projected 2D image to a threshold value) and used to generate an extent annotation 210 that could be indicated on the overlaid display 200. A region of the target annotated region 105 that does not overlap with the extent annotation 210 could be determined (e.g., to indicate portions of the target represented by the target annotated region 105 that were not removed during the procedure to explant the sample) and provided on the display as a non-overlapping annotation 220. A surgeon or other healthcare professional could then use this indicated non-overlapping annotation 220 to decide whether to remove additional tissue from a patient, and if so, along which margin of the explantation site to remove such tissue from.

Additional or alternative analyses and related indications could be provided based on sets of target 2D images and 3D images and/or projected 2D images registered thereto. For example, features of interest could be identified and located within a target 2D image and related 3D image and/or projected 2D image and indications related thereto provided. This could include identifying a number, location, or other information about calcifications or other tumor-related features of interest within the image data and then providing an indicated related thereto. Such an indication could include an indication of a number of calcifications identified in the pre-surgical target 2D image and an indication of a number calcifications identified in the 3D image (or projected 2D image determined therefrom) of an explanted sample. A surgeon could use such indications to determine whether to remove additional tissue (e.g., if the number of calcifications identified within the pre-surgical target 2D image is less than the number of calcifications identified within the explanted sample). In some examples, the image data could be used to determine correspondences between individual identified calcifications within the pre-surgical target 2D image and individual calcifications identified within the imagery of the explanted sample. Indications could then be provided related to such correspondences, e.g., lines or other indications on a display that link corresponding calcifications in the pre-surgical and explanted sample imagery, indications of calcifications within the pre-surgical imagery for which no corresponding calcification was identified within the explanted sample imagery, or some other indications.

The particular perspective views of sample provided as provided in a display as described herein can be controlled by a user in a variety of ways. For example, a user could click and drag to rotate the perspective view about an axis, or use a two-finger gesture to zoom in or out. Alternatively, buttons arranged as a quartet of directional arrows or some other user interface element (not shown) could be used to accomplish such changes. The type of perspective view (e.g., surface coloration according to orientation, projected density view with internal structures indicated, etc.) could be modified by pressing buttons on the user interface, engaging a drop-down menu, or by some other means. For example, the user interface could be used (e.g., by clicking or otherwise interacting with a button, not shown) to switch between a simulated surface render view to a simulated slice view.

An imaging apparatus used to generate volumetric density information or other 3D image data as described herein could include a variety of components to facilitate a variety of different volumetric imaging modalities. In some examples, the imager could include high-power magnets (e.g., superconducting magnets), bias coils, radiofrequency scan coils, and other elements configured to perform magnetic resonance imaging (MRI) of the sample. Such an MRI imager could generate volumetric density information for the target sample related to the density of hydrogen atoms, MRI contrast medium atoms (e.g., Gadolinium), or related to the density of some other magnetic particle. In some examples, the imager could include a micro-CT imager configured to generate volumetric density information for the target sample related to the X-ray radiodensity or radiopacity of the sample.

Such a micro-CT imager includes at least one X-ray source, capable of generating X-rays, and at least one X-ray imager, capable of generating images of the emitted X-rays after having passed through the target sample. Higher-density regions of the target sample (which may alternatively be referred to as regions having higher X-ray radiodensity or radiopacity) will absorb and/or scatter the emitted X-rays to a greater degree, resulting in corresponding regions of the X-ray imager being exposed to a lower intensity of X-rays. A micro-CT imager operates to generate scan data in the form of a plurality of X-ray images of a target sample, each image taken at a respective angle and/or location relative to the target sample. The plurality of X-ray images of a target sample can then be reconstructed to generate volumetric density information for the target sample.

The X-ray source could include an X-ray tube, a cyclotron, a synchrotron, a radioactive X-ray source, or some other source of X-rays. The X-ray source could include multiple different sources of X-rays, e.g., to permit modulation of the beam power, beam width, the direction of the X-ray beam relative to a target sample, a focus or divergence of the X-ray beam at the location of a target sample, or to allow control of some other property of the emitted X-rays so as to facilitate imaging of a target sample.

The X-ray imager could include a photostimulable phosphor plate, scintillator, X-ray intensifier, or other element to convert X-rays into visible light coupled to a charge-coupled device, array of photodetectors, flat-panel detectors, or other visible-light imaging element(s). Additionally or alternatively, the X-ray imager could include an amorphous selenium element or some other element configured to convert X-rays directly into electron-hole pairs or other electronically-detectable phenomena. The X-ray imager and X-ray source together define a field of view, which is a region that the micro-CT imager can image. Thus, the micro-CT imager can generate an X-ray image of portions of a target sample (or other substances or structures) that are located within the field of view.

Micro-CT imaging of samples that have been removed from a body allows for the use of higher-intensity and longer-duration scans than would be possible when imaging parts of a living patient's body. Additionally, the X-ray source and X-ray imager can be located closer to the sample. These factors contribute to increased image resolution and contrast when compared to imaging tissues located within a patient's body. Further, the location and orientation of an explanted tissue sample can be arbitrarily rotated and/or translated by an actuated gantry, allowing the exact location and orientation of the sample relative to the imaging apparatus to be arbitrarily and precisely controlled. For example, X-ray images can be taken of the sample at non-uniform angles or some other reduced or sparse set of angles. Additionally, when the entire sample is small enough to fit entirely within the field of view of the imaging apparatus, the actuated gantry can be operated to ensure that the sample is, in fact, located entirely within the field of view. In some examples, a sample receptacle configured to contain the sample could have a size that is approximately coextensive with the field of view, ensuring that any sample deposited therein will remain entirely within the field of view. Alternatively, when the sample is too large to fit entirely within the field of view, the location and orientation of the sample can be controlled to obtain X-ray images at specific relative locations and orientations sufficient to allow reconstruction of volumetric density information for the entire sample.

Imaging of explanted tissue samples also allows the X-ray source to be entirely enclosed within X-ray shielding material (e.g., lead sheeting) when the X-ray source is being operated to emit X-rays. For example, a door composed of X-ray shielding material could be translated and/or rotated into place after the sample has been deposited within the micro-CT imager, reducing the amount of X-ray exposure experienced by surgeons, nurses, or other persons in proximity to the imager. This can also allow the intensity of X-rays emitted by the X-ray source to be increased while maintaining environmental exposure limits below a specified safe level, potentially increasing image resolution and/or contrast.

A micro-CT imager used to generate volumetric density information as described herein could be operated in a variety of ways to generate X-ray scan data of a sample sufficient to generate an accurate reconstruction of volumetric density information for the sample. The reconstruction methods described in U.S. Pat. No. 8,605,975, U.S. application no. 2014/0161332, U.S. Pat. Nos. 9,189,871, 9,613,442, PCT application no. US18/52175, and U.S. Provisional Patent Application No. 62/562,138 allow for accurate reconstruction of such volumetric density information using a reduced number of X-ray images of a sample relative to other methods. In particular, the reduced view and sparse view reconstruction methods described in those patents and patent applications, permit the generation of clinical-quality volumetric density information for explanted breast tissue or other target tissue sample using less than 300 individual X-ray images of the sample, or less than 100 individual X-ray images of the sample. This reduction in the number of X-ray images needed for reconstruction can lead to a reduction in the overall scan time to less than ten minutes, or less than 5 minutes.

Such an imaging system can be configured to create volumetric density information for a sample using a micro-CT imager or other X-ray based tomographic technology. However, such an imaging system could include additional or alternative imaging technologies, e.g., magnetic resonance imaging, volumetric fluorescence imaging, ultrasound imaging, far-ultraviolet imaging, spontaneous emission imaging (e.g., positron-emission imaging), or some other form of volumetric imaging, or some combination of modalities. Indeed, precise automated specimen handling described herein (e.g., using standardized sample receptacles with registration features and/or imaging fiducials) could facilitate the automated imaging of a sample using multiple imaging modalities. The lack of human intervention in the sample handling between imaging modalities could improve registration of data from multiple different imaging modalities by reducing the amount of sample motion or deformation that may occur between performances of the multiple different imaging modalities.

### III. EXAMPLE SYSTEMS

Computational functions described herein may be performed by one or more computing systems. Such computational functions may include functions to operate an imager to generate scan data for a target sample, functions to reconstruct volumetric density information from such scan data, functions to render cross-sectional, perspective, numerically projected, or other two-dimensional views from the volumetric density data, functions to register or otherwise align such three-dimensional density data (or two-dimensional projections thereof) to two- or three-dimensional image data generated by some other system (e.g., by a mammographic imaging system), and/or user interface functions. Such a computing system may be integrated into or take the form of a computing device, such as a portable medical imaging system, a remote interface for such an imaging system, a pathologist's workstation, a tissue analysis and/or sectioning table or workstation, a tablet computer, a laptop computer, a server, a cloud computing network, and/or a programmable logic controller.

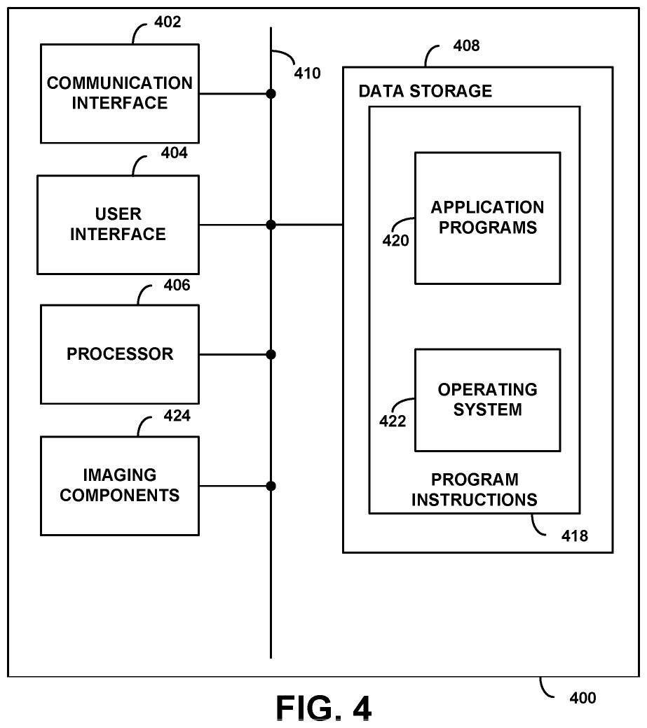

For purposes of example, FIG. 4 is a simplified block diagram showing some of the components of an example computing device 400 that may include components for providing indications of scan-related data onto screen or other display device. Alternatively, an example computing device may lack such components and provide indications of imaging data via some other means (e.g., via the internet or some other network or other communications interface).

The computing device 400 may also include imaging components 424 for obtaining imaging data for such a tissue sample. Imaging components 424 may include a micro-CT imager, an MRI imager, and/or some other components configured to provide information indicative of volumetric density information or other types of 3D image data (e.g., 3D tensors indicative of the pattern of diffusion throughout an organ) for a sample. Alternatively, an example computing device may lack such components and receive scan information via some other means (e.g., via the internet or some other network or other communications interface).

As shown in FIG. 4, computing device 400 may include a communication interface 402, a user interface 404, a processor 406, data storage 408, and imaging components 424, all of which may be communicatively linked together by a system bus, network, or other connection mechanism 410.

Communication interface 402 may function to allow computing device 400 to communicate, using analog or digital modulation of electric, magnetic, electromagnetic, optical, or other signals, with other devices, access networks, and/or transport networks. Thus, communication interface 402 may facilitate circuit-switched and/or packet-switched communication, such as plain old telephone service (POTS) communication and/or Internet protocol (IP) or other packetized communication. For instance, communication interface 402 may include a chipset and antenna arranged for wireless communication with a radio access network or an access point. Also, communication interface 402 may take the form of or include a wireline interface, such as an Ethernet, Universal Serial Bus (USB), or High-Definition Multimedia Interface (HDMI) port. Communication interface 402 may also take the form of or include a wireless interface, such as a Wi-Fi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 402. Furthermore, communication interface 402 may comprise multiple physical communication interfaces (e.g., a Wi-Fi interface, a BLUETOOTH® interface, and a wide-area wireless interface).

In some embodiments, communication interface 402 may function to allow computing device 400 to communicate, with other devices, remote servers, access networks, and/or transport networks. For example, the communication interface 402 may function to transmit and/or receive an indication of image information, to transmit an indication of imaging-related data that can then be displayed, to transmit an indication of a relative orientation and/or translation of 3D image data relative to target 2D and/or 3D image data, or some other information. For example, the computing device 400 could be a pathologist's workstation located in a pathologist's office, remote from one or more operating rooms wherein sample explantation and imaging occur, and the remote system could be a display or other system configured to display the results of analyses as described herein to facilitate the diagnosis and treatment of disease by surgeons in the operating room(s).

In some examples, the computing device 400 could include a volumetric imaging system (e.g., a micro-CT imager) and computational resources for reconstructing volumetric density information or other types of 3D images from scan data, for identifying regions of interest from the volumetric density information, for registering the 3D images to target 2D and/or 3D images (e.g., 2D mammogram images), for rendering images of tissue samples based on the volumetric density information (e.g., perspective views, simulated two-dimensional slices through the sample, numerically-generated simulated 2D images through the sample as projected onto a specified 2D plane, etc.), or for performing some other computational tasks. Such computational resources could include one or more GPUs or other processors specialized for reconstruction, rendering, or other image-processing tasks as described herein. Such a computing device 400 could be in communication with a terminal device (e.g., a workstation, a tablet computer, a head-mounted display, an automated sectioning tool, a thin client) and could provide rendered images to such a terminal in response to user inputs indicative of such rendered images. For example, a user input to a user interface (e.g., keyboard, touchscreen, mouse, head tracker of a head-mounted display) could cause the terminal device to send, to the computing device 400, a request for imaging data related to the user input (e.g., a request for an updated two-dimensional numerical projection of the 3D density information image based on a user input updating the registered relative orientation and/or location of the 3D density information relative to a target 2D or 3D image). The computing device 400 could then, in response to the request, transmit to the terminal device some information indicative of the requested data (e.g., one or more two-dimensional images, a wireframe/segmentation map or other simplified representation of the volumetric density information or other 3D image data). Such operations could allow the terminal device to be lower cost, lighter, smaller, or otherwise improved to facilitate interaction therewith by a pathologist or other healthcare professional while maintaining access to the imaging and processing resources of the computing device 400.

User interface 404 may function to allow computing device 400 to interact with a user, for example to receive input from and/or to provide output to the user. Thus, user interface 404 may include input components such as a keypad, keyboard, touch-sensitive or presence-sensitive panel, computer mouse, trackball, joystick, microphone, and so on. User interface 404 may also include one or more output components such as a display screen which, for example, may be combined with a presence-sensitive panel. The display screen may be based on CRT, LCD, and/or LED technologies, or other technologies now known or later developed. User interface 404 may also be configured to generate audible output(s), via a speaker, speaker jack, audio output port, audio output device, earphones, and/or other similar devices.

In some embodiments, user interface 404 may include a display that serves to provide, indications of 2D and/or 3D images, potentially overlaid on each other (e.g., a numerically-generated 2D projection of a 3D image that has been aligned to a target 2D image), regions of interest within such images, or other imaging-related information to a user. Additionally, user interface 404 may include one or more buttons, switches, knobs, and/or dials that facilitate the configuration and operation of the imaging components 424 or to configure some other operation of the computing device 400. It may be possible that some or all of these buttons, switches, knobs, and/or dials are implemented as functions on a touch- or presence-sensitive panel.

Processor 406 may comprise one or more general purpose processors—e.g., microprocessors—and/or one or more special purpose processors—e.g., digital signal processors (DSPs), graphics processing units (GPUs), floating point units (FPUs), network processors, or application-specific integrated circuits (ASICs). In some instances, special purpose processors may be capable of image processing, image registration and/or scaling, tomographic reconstruction, numerical simulation of 2D projection images from 3D image data, among other applications or functions. Data storage 408 may include one or more volatile and/or non-volatile storage components, such as magnetic, optical, flash, or organic storage, and may be integrated in whole or in part with processor 406. Data storage 408 may include removable and/or non-removable components.

Processor 406 may be capable of executing program instructions 418 (e.g., compiled or non-compiled program logic and/or machine code) stored in data storage 408 to carry out the various functions described herein. Therefore, data storage 408 may include a non-transitory computer-readable medium, having stored thereon program instructions that, upon execution by computing device 400, cause computing device 400 to carry out any of the methods, processes, or functions disclosed in this specification and/or the accompanying drawings.

By way of example, program instructions 418 may include an operating system 422 (e.g., an operating system kernel, device driver(s), and/or other modules) and one or more application programs 420 (e.g., sample scanning functions, reconstruction or rendering functions) installed on computing device 400.

Application programs 420 may take the form of “apps” that could be downloadable to computing device 400 through one or more online application stores or application markets (via, e.g., the communication interface 402). However, application programs can also be installed on computing device 400 in other ways, such as via a web browser or through a physical interface (e.g., a USB port) of the computing device 400.

In some examples, portions of the methods described herein could be performed by different devices, according to an application. For example, different devices of a system could have different amounts of computational resources (e.g., memory, processor cycles) and different information bandwidths for communication between the devices. For example, a first device could be a pathologist's workstation or remote interface that could transmit commands and/or requests for imaging data to another device or server that has the necessary computational resources to perform the reconstruction and/or rendering methods required to generate the requested imaging data, e.g., from CT scan data of a tissue sample. Different portions of the methods described herein could be apportioned according to such considerations.

### IV. EXAMPLE METHODS

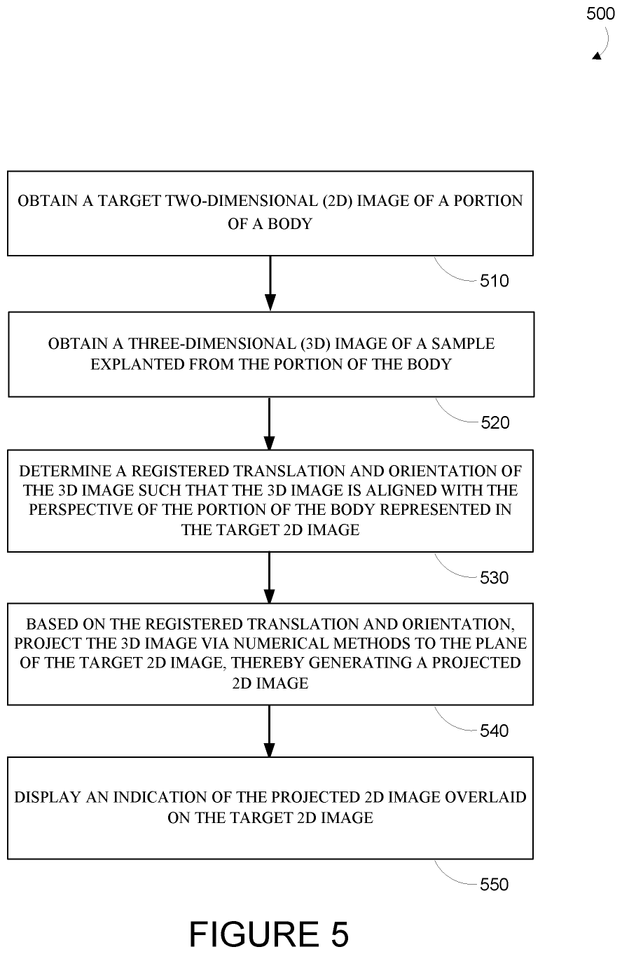

FIG. 5 is a flowchart of a method 500. The method 500 includes obtaining a target two-dimensional (2D) image of a portion of a body (510). The method 500 additionally includes obtaining a three-dimensional (3D) image of a sample explanted from the portion of the body (520). The method 500 also includes determining a registered translation and orientation of the 3D image such that the 3D image is aligned with the perspective of the portion of the body represented in the target 2D image (530). The method 500 yet further includes based on the registered translation and orientation, projecting the 3D image via numerical methods to the plane of the target 2D image, thereby generating a projected 2D image (540). The method 500 also includes displaying an indication of the projected 2D image overlaid on the target 2D image (550). The method 500 could include additional elements or features.

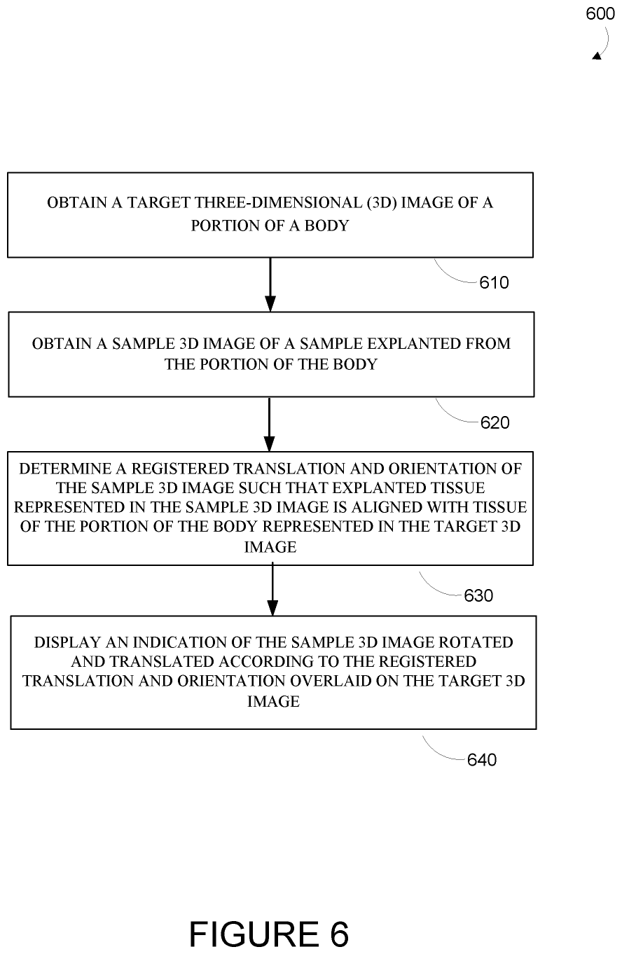

FIG. 6 is a flowchart of a method 600. The method 600 includes obtaining a target three-dimensional (3D) image of a portion of a body (610). The method 600 additionally includes obtaining a sample 3D image of a sample explanted from the portion of the body (620). The method 600 also includes determining a registered translation and orientation of the sample 3d image such that explanted tissue represented in the sample 3d image is aligned with tissue of the portion of the body represented in the target 3d image (630). The method 600 also includes displaying an indication of the sample 3D image rotated and translated according to the registered translation and orientation overlaid on the target 3D image (640). The method 600 could include additional elements or features.

In any of the methods described herein (e.g., methods 500, 600, or other embodiments described herein), the process of obtaining (e.g., “receiving”) volumetric density information or other 2D and/or 3D image information about a target sample and/or region of a body could include a variety of different processes and/or apparatus. In some examples, the image information could be stored on a hard drive that is accessed to and used according to the embodiments described herein. Such stored image information could be generated near in time and/or space to its use to facilitate guidance of surgical procedures (e.g., explantation of samples of tissue in order to, e.g., remove a tumor or other target) or could be generated a longer period of time before and/or distance away from the time and place at which the information is used to facilitate diagnosis of a condition, planning or provision of a treatment (e.g., a follow-up tissue removal surgery), or some other end. For example, the image data could be generated by operating an X-ray scanner or other volumetric imaging device that is located in an operating room where the tissue sample is removed from a patient. Such volumetric density information could be used by a surgeon and/or radiologist to decide, during the tissue removal procedure, whether additional tissue should be removed from the patient and, if so, from what location(s) within the patient's body.

### V. CONCLUSION

The above detailed description describes various features and functions of the disclosed systems, devices, and methods with reference to the accompanying figures. In the figures, similar symbols typically identify similar components, unless context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The embodiments herein are described as being used by pathologists, radiologists, surgeons, and other healthcare professionals to facilitate sectioning or other manipulation or analysis of tissue samples in an image-guided manner and to visualize such image data to select planes through which to section such samples or to otherwise target further manipulations and/or analysis of such samples. However, these are merely illustrative example applications. The embodiments described herein could be employed to image, section, or otherwise manipulate other objects or substances of interest (e.g., plant or animal tissue) and to visualize such image data.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

### VI. ENUMERATED EXAMPLE EMBODIMENTS

Embodiments of the present disclosure may thus relate to one of the enumerated example embodiments (EEEs) listed below. It will be appreciated that features indicated with respect to one EEE can be combined with other EEEs.

EEE 1 is a method including: (i) obtaining a target two-dimensional (2D) image of a portion of a body; (ii) obtaining a three-dimensional (3D) image of a sample explanted from the portion of the body; (iii) determining a registered translation and orientation of the 3D image such that the 3D image is aligned with the perspective of the portion of the body represented in the target 2D image; (iv) based on the registered translation and orientation, projecting the 3D image via numerical methods to the plane of the target 2D image, thereby generating a projected 2D image; and (v) displaying an indication of the projected 2D image overlaid on the target 2D image.

EEE 2 is the method of EEE 1, wherein determining the registered translation and orientation includes: (i) for each candidate orientation of a plurality of candidate orientations of the 3D image, rotating the 3D image according to the candidate orientation and projecting the rotated 3D image via numerical methods to generate a candidate projected 2D image; (ii) for each candidate orientation of the plurality of candidate orientations, determining a candidate translation of the candidate projected 2D image to maximize a similarity metric between the target 2D image and the candidate projected 2D image, thereby generating a respective similarity score for the candidate orientation and translation; and (iii) determining a maximum similarity score of the similarity scores determined for the plurality of candidate orientations and translations and selecting the corresponding candidate orientation and translation as the registered translation and orientation.

EEE 3 is the method of EEE 2, wherein the similarity metric is a mutual information between the target 2D image and the candidate projected 2D image.

EEE 4 is the method of EEE 2, further including: discarding pixels of the target 2D image and candidate projected 2D image that do not exceed a threshold intensity, wherein determining the candidate translation of the candidate projected 2D image to maximize the similarity metric between the target 2D image and the candidate projected 2D image comprises determining the candidate translation of the candidate projected 2D image to maximize the similarity metric between the non-discarded pixels of the target 2D image and the non-discarded pixels of the candidate projected 2D image.

EEE 5 is the method of EEE 4, wherein the threshold intensity is specified such that pixels of the target 2D image and candidate projected 2D image are discarded that do not depict metallic matter, ceramic matter, synthetic polymeric matter, or radiopaque matter.

EEE 6 is the method of EEE 4, wherein the threshold intensity is specified such that pixels of the target 2D image and candidate projected 2D image are discarded that do not depict metallic matter, ceramic matter, synthetic polymeric matter, radiopaque matter, or calcifications.

EEE 7 is the method of any preceding EEE, wherein determining the registered translation and orientation includes: (i) determining, based on the 3D image, an orientation and location of a fiducial within the 3D image, wherein the fiducial has a geometry that is rotationally non-degenerate; (ii) determining, based on the target 2D image, an orientation and location of the fiducial within the target 2D image; and (iii) determining the registered translation and orientation of the 3D image based on a difference between the orientation and location of the fiducial within the 3D image and the orientation and location of the fiducial within the target 2D image.

EEE 8 is the method of any preceding EEE, wherein determining the registered translation and orientation includes: (i) determining, based on the target 2D image and the 3D image, a plurality of candidate translations and orientations of the 3D image to align the 3D image with the perspective of the portion of the body represented in the target 2D image; (ii) generating, via numerical methods for each of the candidate translations and orientations, candidate projected 2D images of the 3D image projected to the plane of the target 2D image; (iii) displaying an indication of the candidate projected 2D images overlaid on the target 2D image; (iv) receiving a user selection of one of the candidate projected 2D images; and (v) determining the registered translation and orientation of the 3D image based on the candidate translation and orientation that corresponds to the selected candidate projected 2D image.

EEE 9 is the method of any preceding EEE, wherein determining the registered translation and orientation includes: (i) determining, based on the target 2D image and the 3D image, a candidate translation and orientation of the 3D image to align the 3D image with the perspective of the portion of the body represented in the target 2D image; (ii) generating, via numerical methods, a candidate projected 2D image of the 3D image projected to the plane of the target 2D image; (iii) displaying an indication of the candidate projected 2D image overlaid on the target 2D image; (iv) receiving a user modification of a candidate translation and orientation; and (v) determining the registered translation and orientation of the 3D image based on the user modification of the candidate translation and orientation.

EEE 10 is the method of any preceding EEE, further including: (i) identifying and counting a plurality of calcifications within the target 2D image; (ii) identifying and counting a plurality of calcifications within the projected 2D image; and (iii) displaying an indication of at least one of (a) the count of calcifications within the target 2D image and the count of calcifications within the target 2D image or (b) a set of correspondences between individual calcifications within the target 2D image and individual calcifications within the projected 2D image.

EEE 11 is the method of any preceding EEE, further including: i) obtaining annotation information for at least one of the target 2D image, the 3D image, or the projected 2D image, wherein displaying the indication of the projected 2D image overlaid on the target 2D image comprises displaying an indication of the annotation information overlaid on the projected 2D image and target 2D image.

EEE 12 is the method of EEE 11, wherein obtaining the annotation information comprises determining, based on the 3D image, a segmentation map of one or more volumes of interest within the 3D image.

EEE 13 is the method of EEE 11 or 12, wherein obtaining the annotation information comprises obtaining an indication of the extent of a region of interest within the target 2D image, and wherein the method further comprises: (i) determining a location and extent of a remnant portion of the region of interest that extends beyond the extent of the explanted sample as depicted in the projected 2D image; and (ii) wherein displaying the indication of the projected 2D image overlaid on the target 2D image comprises displaying an indication of the location and extent of the remnant portion overlaid on the projected 2D image and target 2D image.

EEE 14 is the method of any preceding EEE, wherein determining the registered translation and orientation and projecting the 3D image via numerical methods to the plane of the target 2D image are performed by a controller of a system that also comprises an imager that is operable to image samples of interest, and wherein obtaining the 3D image of the explanted sample comprises the controller operating the imager to image the explanted sample, thereby generating the 3D image.

EEE 15 is the method of EEE 14, further including: transmitting, by the controller to a remote system, an indication of the projected 2D image.

EEE 16 is the method of EEE 14 or 15, wherein displaying the indication of the projected 2D image overlaid on the target 2D image is performed by a display of the system while the explanted sample is located within the imager.

EEE 17 is the method of EEE 14, 15, or 16, wherein the imager is a micro-computed tomography (CT) imager comprising an X-ray source, an X-ray imager, and a sample receptacle configured to contain the explanted sample, wherein the X-ray source and the X-ray imager define a field of view, and wherein operating the imager to generate scan data for the target sample comprises rotating the sample receptacle and operating the X-ray source and the X-ray imager to generate a plurality of X-ray images of the target sample.

EEE 18 is a method including: (i) obtaining a target three-dimensional (3D) image of a portion of a body; (ii) obtaining a sample 3D image of a sample explanted from the portion of the body; (iii) determining a registered translation and orientation of the sample 3D image such that explanted tissue represented in the sample 3D image is aligned with tissue of the portion of the body represented in the target 3D image; and (iv) displaying an indication of the sample 3D image rotated and translated according to the registered translation and orientation overlaid on the target 3D image.

EEE 19 is the method of EEE 18, wherein determining the registered translation and orientation includes: (i) for each candidate orientation of a plurality of candidate orientations of the sample 3D image, rotating the sample 3D image according to the candidate orientation and determining a candidate translation of the rotated 3D image to maximize a similarity metric between the target 3D image and the rotated 3D image, thereby generating a respective similarity score for the candidate orientation and translation; and (ii) determining a maximum similarity score of the similarity scores determined for the plurality of candidate orientations and translations and selecting the corresponding candidate orientation and translation as the registered translation and orientation.

EEE 20 is the method of EEE 19, wherein the similarity metric is a mutual information between the rotated 3D image and the target 3D image.

EEE 21 is the method of EEE 19, further including: discarding pixels of the target 3D image and rotated 3D image that do not exceed a threshold intensity, wherein determining the candidate translation of the rotated 3D image to maximize the similarity metric between the target 3D image and the rotated 3D image comprises determining the candidate translation of the rotated 3D image to maximize the similarity metric between the non-discarded pixels of the target 3D image and the non-discarded pixels of the rotated 3D image.

EEE 22 is the method of EEE 21, wherein the threshold intensity is specified such that pixels of the target 3D image and rotated 3D image are discarded that do not metallic matter, ceramic matter, synthetic polymeric matter, or radiopaque matter.

EEE 23 is the method of EEE 21, wherein the threshold intensity is specified such that pixels of the target 3D image and rotated 3D image are discarded that do not depict metallic matter, ceramic matter, synthetic polymeric matter, radiopaque matter, or calcifications.

EEE 24 is the method of any of EEEs 18-23, wherein determining the registered translation and orientation comprises: (i) determining, based on the sample 3D image, an orientation and location of a fiducial within the sample 3D image, wherein the fiducial has a geometry that is rotationally non-degenerate; (ii) determining, based on the target 3D image, an orientation and location of the fiducial within the target 3D image; and (iii) determining the registered translation and orientation of the sample 3D image based on a difference between the orientation and location of the fiducial within the sample 3D image and the orientation and location of the fiducial within the target 3D image.

EEE 25 is the method of any of EEEs 18-24, wherein determining the registered translation and orientation includes: (i) determining, based on the target 3D image and the sample 3D image, a plurality of candidate translations and orientations of the sample 3D image to align the sample 3D image with the perspective of the portion of the body represented in the target 3D image; (ii) generating candidate registered 3D images of the sample 3D image rotated and translated according to each of the candidate translations and orientations; (iii) displaying an indication of the candidate registered 3D images overlaid on the target 3D image; (iv) receiving a user selection of one of the candidate registered 3D images; and (v) determining the registered translation and orientation of the sample 3D image based on the candidate translation and orientation that corresponds to the selected candidate registered 3D image.

EEE 26 is the method of any of EEEs 18-25, wherein determining the registered translation and orientation includes: (i) determining, based on the target 3D image and the sample 3D image, a candidate translation and orientation of the sample 3D image to align the sample 3D image with the perspective of the portion of the body represented in the target 3D image; (ii) generating a candidate registered 3D image of the sample 3D image rotated and translated according to the candidate rotation and translation; (iii) displaying an indication of the candidate registered 3D image overlaid on the target 3D image; (iv) receiving a user modification of the candidate translation and orientation; and (v) determining the registered translation and orientation of the sample 3D image based on the user modification of the candidate translation and orientation.

EEE 27 is the method of any of EEEs 18-26, further including: (i) identifying and counting a plurality of calcifications within the target 3D image; (ii) identifying and counting a plurality of calcifications within the sample 3D image; and (iii) displaying an indication of at least one of (a) the count of calcifications within the target 3D image and the count of calcifications within the sample 3D image or (b) a set of correspondences between individual calcifications within the target 3D image and individual calcifications within the sample 3D image.

EEE 28 is the method of any of EEEs 18-27, further including: obtaining annotation information for at least one of the target 3D image or the sample 3D image, wherein displaying the indication of the sample 3D image overlaid on the target 3D image comprises displaying an indication of the annotation information overlaid on the sample 3D image and target 3D image.

EEE 29 is the method of EEE 28, wherein obtaining the annotation information comprises determining, based on the sample 3D image, a segmentation map of one or more volumes of interest within the sample 3D image.

EEE 30 is the method of any of EEEs 28-29, wherein obtaining the annotation information comprises obtaining an indication of the extent of a region of interest within the target 3D image, and wherein the method further comprises: (i) determining a location and extent of a remnant portion of the region of interest that extends beyond the extent of the explanted sample as depicted in the sample 3D image; and (ii) wherein displaying the indication of the sample 3D image overlaid on the target 3D image comprises displaying an indication of the location and extent of the remnant portion overlaid on the sample 3D image and target 3D image.

EEE 31 is the method of any of EEEs 18-30, wherein determining the registered translation and orientation is performed by a controller of a system that also comprises an imager that is operable to image samples of interest, and wherein obtaining the sample 3D image of the explanted sample comprises the controller operating the imager to image the explanted sample, thereby generating the sample 3D image.

EEE 32 is the method of EEE 31, wherein displaying the indication of the sample 3D image overlaid on the target 3D image is performed by a display of the system while the explanted sample is located within the imager.

EEE 33 is the method of any of EEEs 31-32, wherein the imager is a micro-computed tomography (CT) imager comprising an X-ray source, an X-ray imager, and a sample receptacle configured to contain the explanted sample, wherein the X-ray source and the X-ray imager define a field of view, and wherein operating the imager to generate scan data for the target sample comprises rotating the sample receptacle and operating the X-ray source and the X-ray imager to generate a plurality of X-ray images of the target sample.

EEE 34 is a non-transitory computer-readable medium, configured to store at least computer-readable instructions that, when executed by one or more processors of a computing device, causes the computing device to perform controller operations to perform the method of any preceding EEE.

EEE 35 is a system including: (i) a controller comprising one or more processors; and (ii) a non-transitory readable medium having stored therein computer-readable instructions that, when executed by the one or more processors of the controller, cause the system to perform the method of any of EEEs 1-33.

1. A method comprising:
obtaining a target two-dimensional (2D) image of a portion of a body;
obtaining a three-dimensional (3D) image of a sample explanted from the portion of the body;
determining a registered translation and orientation of the 3D image such that the 3D image is aligned with the perspective of the portion of the body represented in the target 2D image;
based on the registered translation and orientation, projecting the 3D image via numerical methods to the plane of the target 2D image, thereby generating a projected 2D image; and
displaying an indication of the projected 2D image overlaid on the target 2D image.

2. The method of claim 1, wherein determining the registered translation and orientation comprises:
for each candidate orientation of a plurality of candidate orientations of the 3D image, rotating the 3D image according to the candidate orientation and projecting the rotated 3D image via numerical methods to generate a candidate projected 2D image;
for each candidate orientation of the plurality of candidate orientations, determining a candidate translation of the candidate projected 2D image to maximize a similarity metric between the target 2D image and the candidate projected 2D image, thereby generating a respective similarity score for the candidate orientation and translation; and
determining a maximum similarity score of the similarity scores determined for the plurality of candidate orientations and translations and selecting the corresponding candidate orientation and translation as the registered translation and orientation.

3. The method of claim 2, wherein the similarity metric is a mutual information between the target 2D image and the candidate projected 2D image.

4. The method of claim 2, further comprising:
discarding pixels of the target 2D image and candidate projected 2D image that do not exceed a threshold intensity,
wherein determining the candidate translation of the candidate projected 2D image to maximize the similarity metric between the target 2D image and the candidate projected 2D image comprises determining the candidate translation of the candidate projected 2D image to maximize the similarity metric between the non-discarded pixels of the target 2D image and the non-discarded pixels of the candidate projected 2D image.

5. The method of claim 4, wherein the threshold intensity is specified such that pixels of the target 2D image and candidate projected 2D image are discarded that do not depict metallic matter, ceramic matter, synthetic polymeric matter, or radiopaque matter.

6. The method of claim 4, wherein the threshold intensity is specified such that pixels of the target 2D image and candidate projected 2D image are discarded that do not depict metallic matter, ceramic matter, synthetic polymeric matter, radiopaque matter, or calcifications.

7. The method of claim 1, wherein determining the registered translation and orientation comprises:
determining, based on the 3D image, an orientation and location of a fiducial within the 3D image, wherein the fiducial has a geometry that is rotationally non-degenerate;
determining, based on the target 2D image, an orientation and location of the fiducial within the target 2D image; and
determining the registered translation and orientation of the 3D image based on a difference between the orientation and location of the fiducial within the 3D image and the orientation and location of the fiducial within the target 2D image.

8. The method of claim 1, wherein determining the registered translation and orientation comprises:
determining, based on the target 2D image and the 3D image, a plurality of candidate translations and orientations of the 3D image to align the 3D image with the perspective of the portion of the body represented in the target 2D image;
generating, via numerical methods for each of the candidate translations and orientations, candidate projected 2D images of the 3D image projected to the plane of the target 2D image;
displaying an indication of the candidate projected 2D images overlaid on the target 2D image;
receiving a user selection of one of the candidate projected 2D images; and
determining the registered translation and orientation of the 3D image based on the candidate translation and orientation that corresponds to the selected candidate projected 2D image.

9. The method of claim 1, wherein determining the registered translation and orientation comprises:
determining, based on the target 2D image and the 3D image, a candidate translation and orientation of the 3D image to align the 3D image with the perspective of the portion of the body represented in the target 2D image;
generating, via numerical methods, a candidate projected 2D image of the 3D image projected to the plane of the target 2D image;
displaying an indication of the candidate projected 2D image overlaid on the target 2D image;
receiving a user modification of a candidate translation and orientation; and
determining the registered translation and orientation of the 3D image based on the user modification of the candidate translation and orientation.

10. The method of claim 1, further comprising:
identifying and counting a plurality of calcifications within the target 2D image;
identifying and counting a plurality of calcifications within the projected 2D image; and
displaying an indication of at least one of (i) the count of calcifications within the target 2D image and the count of calcifications within the target 2D image or (ii) a set of correspondences between individual calcifications within the target 2D image and individual calcifications within the projected 2D image.

11. The method of claim 1, further comprising:
obtaining annotation information for at least one of the target 2D image, the 3D image, or the projected 2D image,
wherein displaying the indication of the projected 2D image overlaid on the target 2D image comprises displaying an indication of the annotation information overlaid on the projected 2D image and target 2D image.

12. The method of claim 11, wherein obtaining the annotation information comprises determining, based on the 3D image, a segmentation map of one or more volumes of interest within the 3D image.

13. The method of claim 11, wherein obtaining the annotation information comprises obtaining an indication of the extent of a region of interest within the target 2D image, and wherein the method further comprises:
determining a location and extent of a remnant portion of the region of interest that extends beyond the extent of the explanted sample as depicted in the projected 2D image; and
wherein displaying the indication of the projected 2D image overlaid on the target 2D image comprises displaying an indication of the location and extent of the remnant portion overlaid on the projected 2D image and target 2D image.

14. The method of claim 1, wherein determining the registered translation and orientation and projecting the 3D image via numerical methods to the plane of the target 2D image are performed by a controller of a system that also comprises an imager that is operable to image samples of interest, and wherein obtaining the 3D image of the explanted sample comprises the controller operating the imager to image the explanted sample, thereby generating the 3D image.

15-17. (canceled)

18. A method comprising:
obtaining a target three-dimensional (3D) image of a portion of a body;
obtaining a sample 3D image of a sample explanted from the portion of the body;
determining a registered translation and orientation of the sample 3D image such that explanted tissue represented in the sample 3D image is aligned with tissue of the portion of the body represented in the target 3D image; and
displaying an indication of the sample 3D image rotated and translated according to the registered translation and orientation overlaid on the target 3D image.

19. The method of claim 18, wherein determining the registered translation and orientation comprises:
for each candidate orientation of a plurality of candidate orientations of the sample 3D image, rotating the sample 3D image according to the candidate orientation and determining a candidate translation of the rotated 3D image to maximize a similarity metric between the target 3D image and the rotated 3D image, thereby generating a respective similarity score for the candidate orientation and translation; and
determining a maximum similarity score of the similarity scores determined for the plurality of candidate orientations and translations and selecting the corresponding candidate orientation and translation as the registered translation and orientation.

20. (canceled)

21. The method of claim 19, further comprising:
discarding pixels of the target 3D image and rotated 3D image that do not exceed a threshold intensity,
wherein determining the candidate translation of the rotated 3D image to maximize the similarity metric between the target 3D image and the rotated 3D image comprises determining the candidate translation of the rotated 3D image to maximize the similarity metric between the non-discarded pixels of the target 3D image and the non-discarded pixels of the rotated 3D image.

22-23. (canceled)

24. The method of claim 18, wherein determining the registered translation and orientation comprises:
determining, based on the sample 3D image, an orientation and location of a fiducial within the sample 3D image, wherein the fiducial has a geometry that is rotationally non-degenerate;
determining, based on the target 3D image, an orientation and location of the fiducial within the target 3D image; and
determining the registered translation and orientation of the sample 3D image based on a difference between the orientation and location of the fiducial within the sample 3D image and the orientation and location of the fiducial within the target 3D image.

25-26. (canceled)

27. The method of claim 18, further comprising:
identifying and counting a plurality of calcifications within the target 3D image;
identifying and counting a plurality of calcifications within the sample 3D image; and
displaying an indication of at least one of (i) the count of calcifications within the target 3D image and the count of calcifications within the sample 3D image or (ii) a set of correspondences between individual calcifications within the target 3D image and individual calcifications within the sample 3D image.

28. The method of claim 18, further comprising:
obtaining annotation information for at least one of the target 3D image or the sample 3D image,
wherein displaying the indication of the sample 3D image overlaid on the target 3D image comprises displaying an indication of the annotation information overlaid on the sample 3D image and target 3D image, wherein obtaining the annotation information comprises obtaining an indication of the extent of a region of interest within the target 3D image, and wherein the method further comprises:
determining a location and extent of a remnant portion of the region of interest that extends beyond the extent of the explanted sample as depicted in the sample 3D image; and
wherein displaying the indication of the sample 3D image overlaid on the target 3D image comprises displaying an indication of the location and extent of the remnant portion overlaid on the sample 3D image and target 3D image.

29-35. (canceled)
